(19) United States
(12) Patent Application Publication
(10) Pub. No.: US 2025/0049732 A1
(43) Pub. Date: Feb. 13, 2025

(54) Transdermal Delivery Systems and Methods for Inhibiting, Stopping, Blocking, or Modulating Glutaminolysis, Glutamine, and Other Glutaminolysis Catalyzing Enzymes Across Multiple Metabolic Pathways in Human Beings and Animals

(71) Applicant: Dennis Sean Stacey, Victoria (CA)

(72) Inventor: Dennis Sean Stacey, Victoria (CA)

(21) Appl. No.: 18/799,803

(22) Filed: Aug. 9, 2024

Related U.S. Application Data
(60) Provisional application No. 63/518,520, filed on Aug. 9, 2023.

(51) Int. Cl. A61K 9/70; A61K 9/00; A61K 9/06; A61K 9/08; A61K 31/198; A61K 47/20; A61K 47/38

(52) U.S. Cl. CPC A61K 9/7084; A61K 9/0014; A61K 9/06; A61K 9/08; A61K 31/198; A61K 47/20; A61K 47/38

(57) ABSTRACT

The present invention relates to novel methods for transdermal delivery of active molecules that inhibit, stop, block, or modulate glutaminolysis, glutamine, and other glutaminolysis catalyzing enzymes across multiple metabolic pathways in human beings and animals applicable for prevention and treatment of various diseases such as cancer, type 2 diabetes, neurological disorders, such as epilepsy, Alzheimer's disease, and Parkinson's disease, autoimmune disorders and inflammatory conditions, metabolic syndromes, etc. The present invention provides various transdermal delivery systems for use in the methods of the present invention, which include creams, sprays, mists, patches, plugs, sub-dermal injections, and any other suitable transdermal delivery systems for delivery of the active molecules.

### PRIORITY

The current application claims a priority to the U.S. provisional patent application Ser. No. 63/518,520 filed on Aug. 6, 2024.

### INCORPORATION BY REFERENCE

All publications, patents, and patent applications mentioned in this specification are herein incorporated by reference in their entirety to the same extent as if each individual publication or patent application was specifically and individually indicated to be incorporated by reference.

### FIELD OF THE INVENTION

The present invention generally relates to transdermal delivery of molecules and transdermal delivery systems and methods. More specifically, the present invention relates to the transdermal delivery of molecules that inhibit, stop, block, or modulate glutaminolysis, glutamine, and other glutaminolysis catalyzing enzymes across multiple metabolic pathways in human beings and animals, utilizing various transdermal delivery systems and methods.

### BACKGROUND OF THE INVENTION

Glutaminolysis and the amino acid Glutamine are involved in multiple metabolic pathways within the bodies of humans and animals. Modulating, intermittently inhibiting, stopping, or blocking glutaminolysis, glutamine, and other glutaminolysis catalyzing enzymes in these pathways has been explored as a potential therapeutic approach for various diseases or physiological states.

For example, disruption of glutaminolysis can selectively target and inhibit the unregulated growth of certain cells that are suffering from respiratory insufficiency and heavily rely on glutamine for their energy production. Additionally, inhibiting, stopping, blocking, or modulating glutaminolysis or and glutamine availability can impact cellular redox balance, protein synthesis, nucleotide synthesis, glucose synthesis, acid-base regulation, and immune cell function, offering a targeted and selective approach to modulate specific disease-related processes.

Traditional and conventional methods of delivery for molecules inhibiting, stopping, or blocking glutaminolysis, glutamine, and other glutaminolysis catalyzing enzymes in these pathways have primarily focused on oral or intravenous administration, which may present limitations and systemic side effects.

Thus, there is a need for overcoming the aforementioned limitations and systemic side effects with novel delivery systems and methods of delivery for molecules inhibiting, stopping, blocking, or modulating glutaminolysis, glutamine, and other glutaminolysis catalyzing enzymes. The present invention addresses the said need by providing methods and systems for delivery of molecules inhibiting, stopping, blocking, or modulating glutaminolysis, glutamine, and other glutaminolysis catalyzing enzymes by uniquely combining various transdermal systems of delivery for delivery of active molecules inhibiting, stopping, blocking, or modulating glutaminolysis, glutamine, and other glutaminolysis catalyzing enzymes.

### SUMMARY OF THE INVENTION

The following listing of embodiments is a nonlimiting statement of various aspects of the invention. Other aspects and variations will be evident in light of the entire disclosure.

An aspect of the present invention provides a method that provides a non-invasive and potentially more effective alternative to oral or intravenous administration, enabling the reduction of various side-effects, increased efficacy, targeted and localized delivery to the site of action, among other potential benefits.

Other aspects of the present invention provide various methods and systems for transdermal delivery of molecules that inhibit, stop, block, or modulate glutaminolysis, glutamine, and other glutaminolysis catalyzing enzymes across multiple metabolic pathways in human beings and animals comprising various transdermal delivery systems, wherein the transdermal delivery systems include creams, sprays, mists, patches, plugs, sub-dermal injections, and any other suitable transdermal delivery systems.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention and are incorporated in and constitute a part of the present invention and, together with the description, serve to explain the principle of the invention.

FIG. 1 illustrates an embodiment of the present invention and provides a schematic diagram of tropical cream medication and transdermal cream medication over a cross-section of skin.

FIG. 2 illustrates an embodiment of the present invention and provides a schematic diagram of transdermal spray or mist.

FIG. 3 illustrates an embodiment of the present invention and provides a schematic diagram of transdermal patch.

FIG. 4 illustrates an embodiment of the present invention and provides a schematic diagram of transdermal or subdermal plug or patch as shown in panels (a) and (b).

FIG. 5 illustrates an embodiment of the present invention and provides a schematic diagram of transdermal or subdermal injections.

FIG. 6 illustrates various embodiments of the present invention and provides schematic diagrams of various methods of transdermal molecule delivery that include (i) injection techniques as shown in: panel (a) jet injections; and (b) powder injections; (ii) microporation techniques as shown in: (c) solid microneedles; (d) hollow microneedles; (e) coated microneedles; and (f) dissolving microneedles; (iii) localized transport regions as shown in (g) electroporation; (h) sonoporation; and (i) ionophoresis.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as basis for teaching one skilled in the art to variously practice the present invention.

All illustrations of the drawings are for the purpose of describing selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises”, “comprising”, “includes”, “including”, “has”, “having”, “contains”, “containing”, “characterized by”, or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment”, “an embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As discussed above, there remains a need in the art for novel delivery systems and methods of delivery for molecules inhibiting, stopping, or blocking glutaminolysis, glutamine, and other glutaminolysis catalyzing enzymes that for overcome the limitations and systemic side effects associated with traditional and conventional methods primarily focused on oral or intravenous administration and delivery of such molecules. The present invention addressed the said need and provides an alternate and unique solution in the form of various methods of transdermal delivery of molecules that inhibit, stop, block, or modulate glutaminolysis, glutamine, and other glutaminolysis catalyzing enzymes across multiple metabolic pathways in human beings and animals, the method comprising the step of transdermal delivery of said molecules comprising transdermal delivery systems, wherein the transdermal delivery systems include creams, sprays, mists, patches, plugs, sub-dermal injections, and any other suitable transdermal delivery systems.

The terms “subject”, “patient”, and “individual” as used herein are used interchangeably herein to refer to a vertebrate, including mammals and humans. A “subject”, “patient”, or “individual” as used herein, includes any animal that exhibits pain that can be treated with the compositions or formulations or systems, and methods contemplated herein, and that includes laboratory animals, farm animals, and domestic animals or pets, non-human primates and human are included.

As used herein, the term “amount” refers to “an amount effective” or “an effective amount” of a cell to achieve a beneficial or desired prophylactic or therapeutic result, including clinical results.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat”, “treatment”, or “treating” embraces at least an amelioration of the symptoms associated with diseases in the patient, where amelioration is used in a broad sense to refer to at least a reduction in the magnitude of a parameter, e.g., a symptom associated with the disease or condition being treated. As such, “treatment” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

As used herein, and unless otherwise specified, the terms “prevent”, “preventing”, and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment”.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. In some embodiments, the engineered cell or pharmaceutical composition comprising said engineered cell of the disclosure is administered in a prophylactically effective amount.

In an embodiment of the present invention, it provides a method for transdermal delivery of molecules that inhibit, stop, block, or modulate glutaminolysis, glutamine, and other glutaminolysis catalyzing enzymes across multiple metabolic pathways in human beings and animals, the method comprising the steps of administering said molecules via a transdermal delivery system, wherein the transdermal delivery system is a cream designed for application to the skin, the cream comprising a topical formulation of the cream for delivery of an active molecule, wherein the topical formulation of the cream comprises permeation enhancers, skin penetration promoters, and moisturizing agents to optimize transdermal absorption, and wherein the active molecule is a molecule that inhibits, stops, blocks, or modulates glutaminolysis, glutamine, and other glutaminolysis catalyzing enzymes across multiple metabolic pathways in human beings and animals.

In another embodiment of the present invention providing a method as disclosed hereinabove, wherein the active molecule comprises specific inhibitors of glutaminolysis, glutamine, and other glutaminolysis catalyzing enzymes including 6-Diazo-5-oxo-L-norleucine referred to as DON.

In another embodiment of the present invention, providing a method as disclosed hereinabove, wherein the permeation enhancers comprise fatty acids, said fatty acids include oleic acid.

In another embodiment of the present invention, providing a method as disclosed hereinabove, wherein the skin penetration promoters comprise agents that enhance the transport of the active molecule through the dermal layers of skin.

In another embodiment of the present invention, providing a method as disclosed hereinabove, wherein the moisturizing agents to optimize transdermal absorption comprises agents to improve skin hydration and facilitate transdermal absorption.

In other words, the present invention provides a cream formulated to facilitate absorption and transport of the inhibiting or modulating molecules across the transdermal layers. The cream formulation may include a combination of permeation enhancers, skin penetration promoters, and moisturizing agents to optimize transdermal absorption. Examples of permeation enhancers include fatty acids (e.g., oleic acid), alcohols (e.g., ethanol), and surfactants (e.g., polysorbate 80).

In an embodiment of the present invention, it provides a method for transdermal delivery of molecules that inhibit, stop, block, or modulate glutaminolysis, glutamine, and other glutaminolysis catalyzing enzymes across multiple metabolic pathways in human beings and animals, the method comprising the steps of administering said molecules via a transdermal delivery system, wherein the transdermal delivery system is a spray or a mist designed for application to the skin, the spray or a mist comprising a liquid formulation of the spray or mist for delivery of an active molecule, wherein the liquid formulation of the spray or mist comprises specific inhibitors, solvents, surfactants, and stabilizers, and wherein the active molecule is a molecule that inhibits, stops, blocks, or modulates glutaminolysis, glutamine, and other glutaminolysis catalyzing enzymes across multiple metabolic pathways in human beings and animals.

In another embodiment of the present invention, providing a method as disclosed hereinabove, wherein the active molecule comprises specific inhibitors of glutaminolysis, glutamine, and other glutaminolysis catalyzing enzymes including 6-Diazo-5-oxo-L-norleucine referred to as DON.

In another embodiment of the present invention, providing a method as disclosed hereinabove, wherein the solvents are selected from a group comprising water, alcohols, and propylene glycol, wherein alcohols include isopropyl alcohol.

In another embodiment of the present invention, providing a method as disclosed hereinabove, wherein the surfactants include sodium lauryl sulfate.

In another embodiment of the present invention, providing a method as disclosed hereinabove, wherein the stabilizers include hydroxypropyl methylcellulose.

In other words, the present invention provides a liquid formulation containing the active molecules which is dispensed as a fine spray or mist onto the skin. The small droplet size promotes rapid absorption and penetration through the dermal barrier. The liquid formulation may include solvents, surfactants, and stabilizers to optimize the spray/mist delivery method. Suitable solvents may include water, alcohols (e.g., isopropyl alcohol), or propylene glycol, while surfactants (e.g., sodium lauryl sulfate) and stabilizers (e.g., hydroxypropyl methylcellulose) can enhance dispersion and stability.

In an embodiment of the present invention, it provides a method for transdermal delivery of molecules that inhibit, stop, block, or modulate glutaminolysis, glutamine, and other glutaminolysis catalyzing enzymes across multiple metabolic pathways in human beings and animals, the method comprising the steps of administering said molecules via a transdermal delivery system, wherein the transdermal delivery system is a transdermal patch system designed for application to the skin for the delivery of an active molecule, wherein the transdermal patch system comprises a backing layer, an adhesive layer, and a reservoir containing the active molecule, and wherein the active molecule is a molecule that inhibits, stops, blocks, or modulates glutaminolysis, glutamine, and other glutaminolysis catalyzing enzymes across multiple metabolic pathways in human beings and animals.

In another embodiment of the present invention, providing a method as disclosed hereinabove, wherein the active molecule comprises specific inhibitors of glutaminolysis, glutamine, and other glutaminolysis catalyzing enzymes including 6-Diazo-5-oxo-L-norleucine referred to as DON.

In another embodiment of the present invention, providing a method as disclosed hereinabove, the transdermal patch system further comprises a rate-controlling membrane to control the release kinetics of the inhibitors from the patch.

In other words, the present invention provides a transdermal patch system that comprises a backing layer, an adhesive layer, and a reservoir containing the inhibiting and/or modulating active molecules. The patch adheres to the skin and releases the molecules continuously or intermittently over a specified period. The patch design may incorporate a rate-controlling membrane or matrix to control the release kinetics. Materials such as polymeric films (e.g., polyethylene), hydrogels (e.g., polyacrylic acid-based hydrogels), or microneedle arrays may be used for the patch structure, ensuring optimal adherence and controlled drug release.

In an embodiment of the present invention, it provides a method for transdermal delivery of molecules that inhibit, stop, block, or modulate glutaminolysis, glutamine, and other glutaminolysis catalyzing enzymes across multiple metabolic pathways in human beings and animals, the method comprising the steps of administering said molecules via a transdermal delivery system, wherein the transdermal delivery system is a plug or patch designed for application to the skin for delivery of an active molecule and is implantable in the skin, wherein the plug or patch comprises biocompatible materials and release active molecules from reservoirs comprising an active molecule, wherein the plug or patch is implanted subcutaneously or subdermally, and wherein the active molecule is a molecule that inhibits, stops, blocks, or modulates glutaminolysis, glutamine, and other glutaminolysis catalyzing enzymes across multiple metabolic pathways in human beings and animals.

In another embodiment of the present invention, providing a method as disclosed hereinabove, wherein the active molecule comprises specific inhibitors of glutaminolysis, glutamine, and other glutaminolysis catalyzing enzymes including 6-Diazo-5-oxo-L-norleucine referred to as DON.

In another embodiment of the present invention, providing a method as disclosed hereinabove, wherein the biocompatible materials are selected from a group comprising biodegradable polymers including polylactic acid, and hydrogels including alginate.

In another embodiment of the present invention, providing a method as disclosed hereinabove, wherein the release reservoir provides controlled release of the active molecule over time.

In other words, the present invention provides a plug or patch, composed of biocompatible materials, which is implanted subcutaneously or subdermally. The plug or patch contains the inhibiting and/or modulating molecules and releases them gradually over time. The plug or patch can be made from biodegradable materials or include reservoirs for controlled molecule release. Biocompatible materials may include biodegradable polymers (e.g., polylactic acid), hydrogels (e.g., alginate), or biodegradable scaffolds. The implantation procedure may involve a minimally invasive technique, such as hypodermic needle insertion or surgical implantation.

In an embodiment of the present invention, it provides a method for transdermal delivery of molecules that inhibit, stop, block, or modulate glutaminolysis, glutamine, and other glutaminolysis catalyzing enzymes across multiple metabolic pathways in human beings and animals, the method comprising the steps of administering said molecules via a transdermal delivery system, wherein the transdermal delivery system is a transdermal injection or subdermal injection designed for application to the skin for delivery of an active molecule, wherein transdermal injection or subdermal injection comprises a delivery device, and wherein the active molecule is a molecule that inhibits, stops, blocks, or modulates glutaminolysis, glutamine, and other glutaminolysis catalyzing enzymes across multiple metabolic pathways in human beings and animals.

In another embodiment of the present invention providing a method as disclosed hereinabove, wherein the active molecule comprises specific inhibitors of glutaminolysis, glutamine, and other glutaminolysis catalyzing enzymes including 6-Diazo-5-oxo-L-norleucine referred to as DON.

In another embodiment of the present invention providing a method as disclosed hereinabove, wherein the delivery device is selected from a group of specialized devices comprising microneedles, cannulas, and biodegradable depots for precise and controlled delivery into the subcutaneous tissue.

In other words, the present invention provides a transdermal or subdermal injection for the delivery of the inhibiting and/or modulating active molecules using a suitable delivery device. This method and delivery system allows direct administration into the subcutaneous tissue of the skin for sustained release. The injection may utilize specialized devices such as microneedles, cannulas, or biodegradable depots to facilitate precise and controlled delivery of the inhibiting molecules. The injection site and depth should be determined based on the desired therapeutic outcome and accessibility.

In an embodiment of the present invention, it provides a method for transdermal delivery of molecules that inhibit, stop, block, or modulate glutaminolysis, glutamine, and other glutaminolysis catalyzing enzymes across multiple metabolic pathways in human beings and animals, the method comprising the steps of administering said molecules via a transdermal delivery system, wherein the transdermal delivery system includes any other suitable transdermal delivery systems and techniques that facilitate the transport and delivery of an active molecule inhibiting, stopping, blocking, or modulating glutaminolysis, glutamine, and other glutaminolysis catalyzing enzymes through the dermal barrier, and wherein the active molecule is a molecule that inhibits, stops, blocks, or modulates glutaminolysis, glutamine, and other glutaminolysis catalyzing enzymes across multiple metabolic pathways in human beings and animals.

In another embodiment of the present invention, providing a method as disclosed hereinabove, wherein the active molecule comprises specific inhibitors of glutaminolysis, glutamine, and other glutaminolysis catalyzing enzymes including 6-Diazo-5-oxo-L-norleucine referred to as DON.

In another embodiment of the present invention, providing a method as disclosed hereinabove, wherein the any other suitable transdermal delivery systems and techniques are selected from a group comprising iontophoresis, electroporation, and sonophoresis.

In embodiments of the present invention as disclosed herein, wherein the methods and systems of transdermal delivery of active molecules of the present invention as disclosed herein are used for treatment and/or prevention and/or management of diseases of humans and animals, the diseases including cancer, diabetes mellitus, neurological disorders, immunological disorders, and metabolic syndromes.

In other words, the present invention provides any other suitable transdermal delivery systems and techniques which may include iontophoresis, electroporation, sonophoresis, or other emerging technologies that enhance transdermal penetration and transport. The selection of the appropriate method depends on the specific properties of the inhibiting/modulating active molecules and the desired release profile.

In the present invention as disclosed, the active molecules that inhibit, stop, block, or modulate glutaminolysis, glutamine, and other glutaminolysis catalyzing enzymes across multiple metabolic pathways work by directly interfering with key enzymes or metabolic processes involved in glutaminolysis, glutamine utilization, and the activity of other glutaminolysis catalyzing enzymes. For example, they may act as competitive inhibitors of glutaminase, the enzyme responsible for catalyzing the conversion of glutamine to glutamate in the glutaminolysis pathway. By inhibiting this enzymatic activity, the molecules disrupt the breakdown of glutamine and subsequently affect downstream metabolic processes such as glutathione synthesis, protein synthesis, nucleotide synthesis, glucose synthesis, acid-base regulation, and immune cell function.

Depending on the transdermal delivery method employed, the components may include cream formulations, liquid formulations for sprays/mists, transdermal patches, plugs/patches, transdermal/subdermal injection devices, or other suitable delivery systems. Each delivery system will have specific components tailored to its functionality, such as permeation enhancers, solvents, stabilizers, adhesive layers, reservoirs, rate-controlling membranes, and biocompatible materials.

The components are put together in a manner that ensures effective transdermal delivery of the inhibiting or modulating molecules. For example, in the case of a cream, the active molecules, such as 6-Diazo-5-oxo-L-norleucine (DON), are incorporated into a suitable base formulation, which may contain permeation enhancers to facilitate their transport across the dermal layers.

The cream is then applied topically to the skin, allowing the inhibiting or modulating molecules to penetrate the skin barrier and reach the underlying tissues. Similarly, in the case of a transdermal patch, the active molecules, such as 6-Diazo-5-oxo-L-norleucine (DON), are contained within a reservoir, which is secured to the skin using an adhesive layer. The patch continuously or intermittently releases the inhibiting or modulating molecules over a specified period, enabling their absorption through the skin and subsequent inhibition or modulation of glutaminolysis, glutamine, or other glutaminolysis catalyzing enzymes in the target metabolic pathways.

In the case of subdermal injection, a delivery device such as a microneedle, cannula, or biodegradable depot is utilized to directly administer the inhibiting or modulating molecules, such as 6-Diazo-5-oxo-L-norleucine (DON), into the subcutaneous tissue. This method ensures sustained release and localized delivery of the molecules, targeting the metabolic pathways where glutaminolysis, glutamine, or other glutaminolysis catalyzing enzyme inhibition or modulation is desired.

The specific formulation, design, and functionality of the components will depend on the intended transdermal delivery method and the desired release kinetics of the inhibiting or modulating molecules.

The methods and systems of transdermal delivery of active molecules of the present invention as disclosed herein can be applied to the treatment of cancer, specifically in line with the work of Dr. Thomas Seyfried, who proposed a metabolic treatment strategy for cancer known as the “Press-Pulse” therapy. The “Press-Pulse” therapy aims to exploit the metabolic differences between normal cells and cancer cells to selectively target cancer cells while preserving normal cells. This may be done as follows:
Step 1—Administer the “press” component, such as a high-fat, caloric restricted, ketogenic diet, to reduce blood glucose to therapeutic levels thereby reducing availability of the fermentable fuel glucose to cancer cells.
Step 2—Administer the “pulse” component by applying the transdermal delivery system and method of the present invention for delivery of the active molecule 6-Diazo-5-oxo-L-norleucine (DON) to the skin in order to inhibit glutaminolysis thereby reducing the availability of the fermentable fuel glutamine to the cancer cell.
Step 3—Administer additional “pulse” components, such as hyperbaric oxygen therapy or other metabolic therapies, to induce metabolic stress in cancer cells.
Step 4—Repeat the press and pulse cycles as necessary to see desired therapeutic effect.

The transdermal delivery systems and methods as disclosed in the present invention hereinabove can be utilized to administer molecules, such as 6-Diazo-5-oxo-L-norleucine (DON), that inhibit, stop, block, or modulate glutaminolysis, glutamine, and other glutaminolysis catalyzing enzymes in cancer cells. This targeted approach disrupts the energy production and proliferation of cancer cells, offering a potential therapeutic strategy for cancer treatment. The process can be applied in conjunction with other cancer treatments or therapies as part of a comprehensive cancer management plan.

The present invention also extends its application to the treatment of various diseases and physiological conditions, beyond cancer. Representative examples of diseases and conditions that can be prevented or treated using the transdermal delivery methods include:

Diabetes Mellitus Type 2—Transdermal delivery of molecules that modulate glutaminolysis in the liver and muscles could regulate glucose metabolism, offering a potential therapeutic approach for managing blood sugar levels in patients with type 2 diabetes.

Neurological Disorders—By inhibiting specific glutaminolysis catalyzing enzymes in the brain, transdermal delivery methods could regulate neurotransmitter levels and potentially alleviate symptoms associated with neurological disorders, such as epilepsy, Alzheimer's disease, and Parkinson's disease.

Immune Disorders—Modulating glutamine utilization and glutaminolysis in immune cells could impact immune response and inflammation, making the transdermal delivery process valuable in treating autoimmune disorders and inflammatory conditions.

Metabolic Syndromes—By targeting glutaminolysis in adipose tissue, transdermal delivery methods could potentially influence lipid metabolism and offer therapeutic benefits in managing metabolic syndromes.

In conclusion, the present invention discloses methods and systems for the transdermal delivery of active molecules that inhibit, stop, block, or modulate glutaminolysis, glutamine, and other glutaminolysis catalyzing enzymes across multiple metabolic pathways in human beings and animals. The methods and systems of the present invention offer several advantages over traditional delivery routes and methods, including improved absorption, targeted delivery, reduced systemic side effects, and enhanced patient compliance. The described embodiments demonstrate the versatility and applicability of the invention across various transdermal delivery methods for the treatment of cancer and other disease states.

The invention will be further explained by the following Examples, which are intended to purely exemplary of the invention, and should not be considered as limiting the invention in any way.

### EXAMPLES

EXAMPLE 1—Exemplary method for transdermal delivery of molecules that inhibit, stop, block, or modulate glutaminolysis, glutamine, and other glutaminolysis catalyzing enzymes across multiple metabolic pathways in human beings and animals

This example provides exemplary embodiments of methods for transdermal delivery of molecules that inhibit, stop, block, or modulate glutaminolysis, glutamine, and other glutaminolysis catalyzing enzymes across multiple metabolic pathways in human beings and animals as disclosed in the present invention.

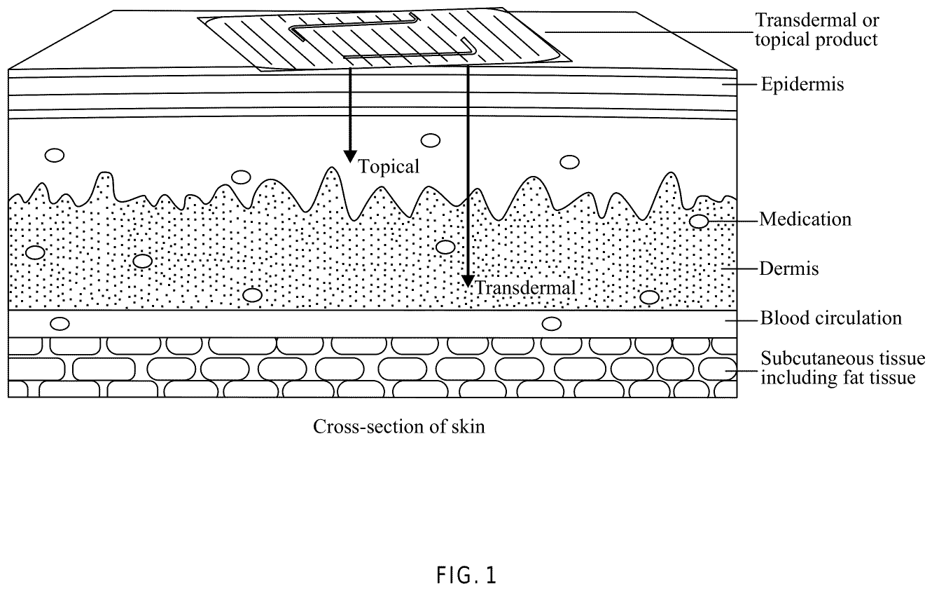

FIG. 1 illustrates first of the exemplary embodiments of methods for transdermal delivery of molecules that inhibit, stop, block, or modulate glutaminolysis, glutamine, and other glutaminolysis catalyzing enzymes across multiple metabolic pathways in human beings and animals administering said molecules via a transdermal delivery system, wherein the transdermal delivery system is a cream of the present invention applied on the skin for topical as well as transdermal delivery of active molecules or medication.

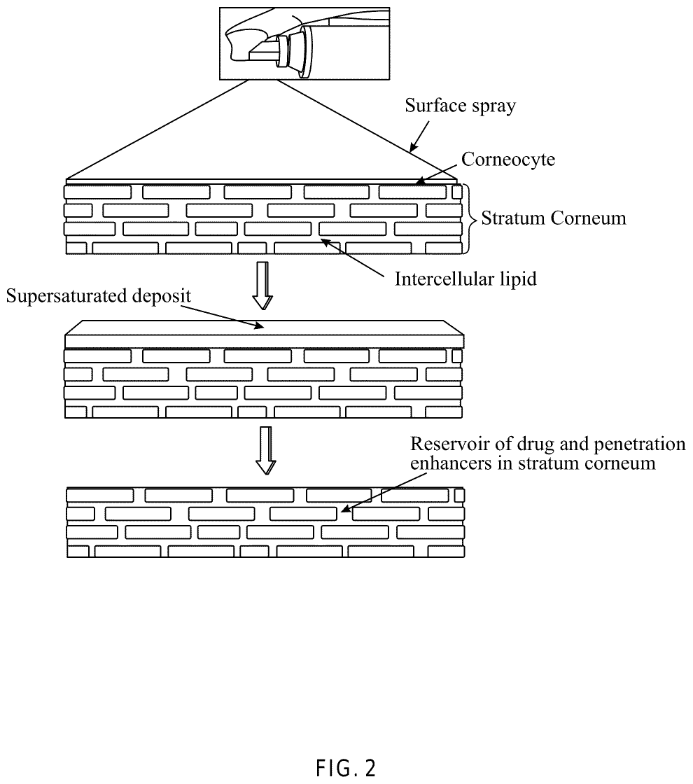

FIG. 2 illustrates second of the exemplary embodiments of methods for transdermal delivery of molecules that inhibit, stop, block, or modulate glutaminolysis, glutamine, and other glutaminolysis catalyzing enzymes across multiple metabolic pathways in human beings and animals administering said molecules via a transdermal delivery system, wherein the transdermal delivery system is a spray or mist of the present invention applied on the skin for transdermal delivery of active molecules or medication.

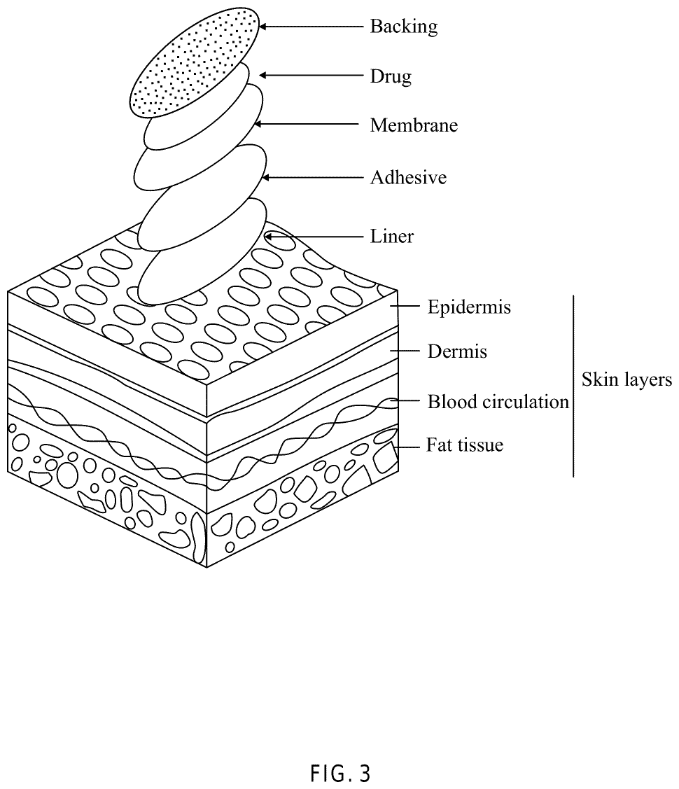

FIG. 3 illustrates third of the exemplary embodiments of methods for transdermal delivery of molecules that inhibit, stop, block, or modulate glutaminolysis, glutamine, and other glutaminolysis catalyzing enzymes across multiple metabolic pathways in human beings and animals administering said molecules via a transdermal delivery system, wherein the transdermal delivery system is a patch of the present invention applied on the skin for transdermal delivery of active molecules or medication.

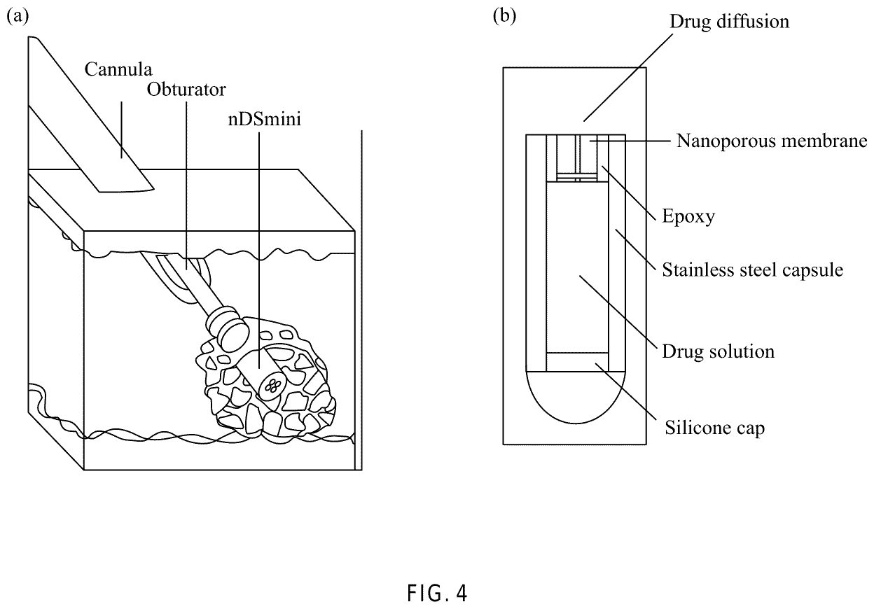

FIG. 4 illustrates fourth of the exemplary embodiments of methods for transdermal delivery of molecules that inhibit, stop, block, or modulate glutaminolysis, glutamine, and other glutaminolysis catalyzing enzymes across multiple metabolic pathways in human beings and animals administering said molecules via a transdermal delivery system, wherein the transdermal delivery system is a transdermal or subdermal patch or plug of the present invention applied on the skin for transdermal delivery of active molecules or medication.

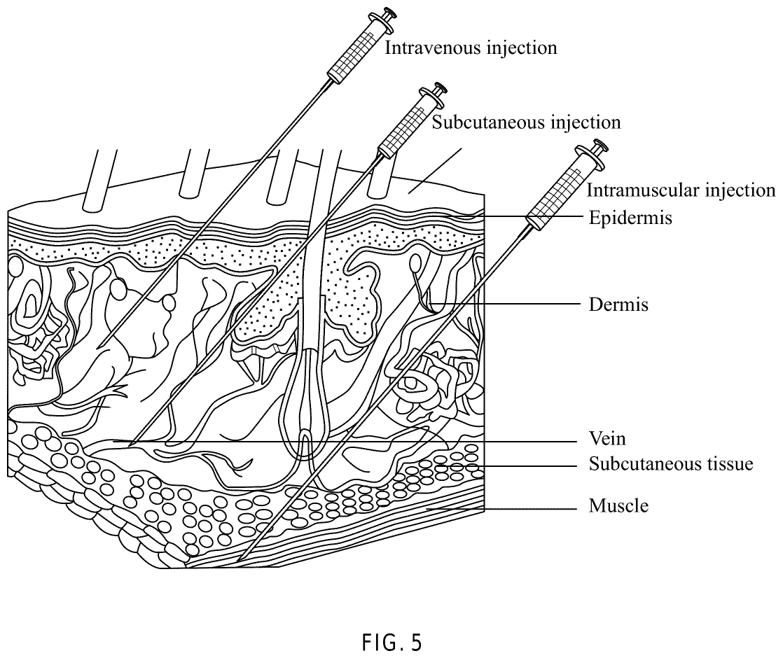

FIG. 5 illustrates exemplary embodiments of various transdermal or subdermal injections of the present invention applied on the skin for transdermal delivery of active molecules or medication delivering it intravenously, subcutaneously, or intramuscularly.

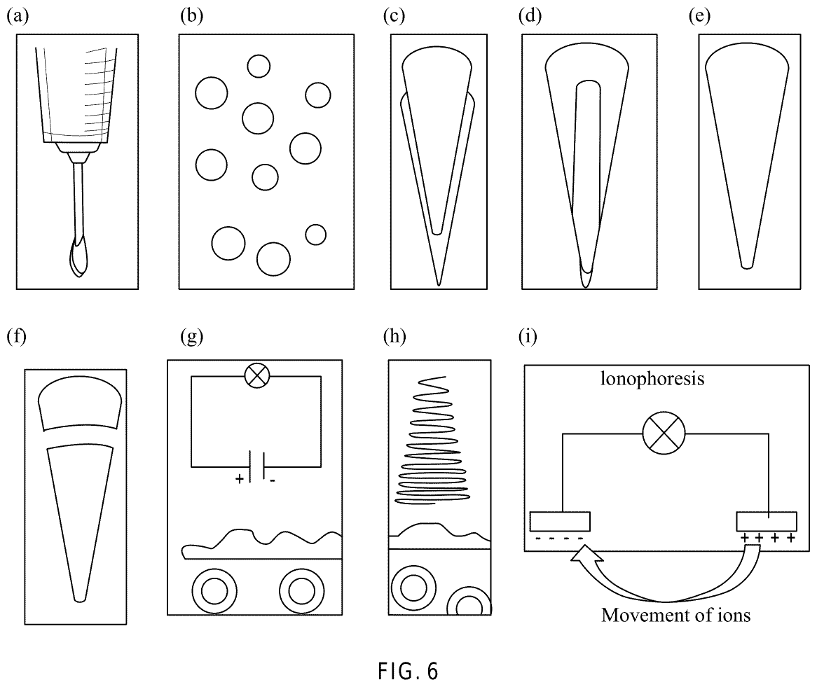

FIG. 6 illustrates exemplary embodiments of various transdermal delivery systems of the present invention for delivery of active molecules or medication including via (a) jet injections, (b) powder injections, (c) solid microneedles; (d) hollow microneedles; (e) coated microneedles; and (f) dissolving microneedles; (g) electroporation; (h) sonoporation; and (i) ionophoresis.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A method for transdermal delivery of molecules that inhibit, stop, block, or modulate glutaminolysis, glutamine, and other glutaminolysis catalyzing enzymes across multiple metabolic pathways in human beings and animals, the method comprising the step of:
administering said molecules via a transdermal delivery system, wherein the transdermal delivery system is a cream designed for application to the skin, the cream comprising a topical formulation of the cream for delivery of an active molecule, wherein the topical formulation of the cream comprises permeation enhancers, skin penetration promoters, and moisturizing agents to optimize transdermal absorption, and wherein the active molecule is a molecule that inhibits, stops, blocks, or modulates glutaminolysis, glutamine, and other glutaminolysis catalyzing enzymes across multiple metabolic pathways in human beings and animals.

2. The method of claim 1, wherein the active molecule comprises specific inhibitors of glutaminolysis, glutamine, and other glutaminolysis catalyzing enzymes including 6-Diazo-5-oxo-L-norleucine referred to as DON.

3. The method of claim 1, wherein the permeation enhancers comprise permeation enhancers selected from a group selected from fatty acids, alcohols, surfactants or a combination thereof.

4. The method of claim 1, wherein the skin penetration promoters comprise agents that enhance the transport of the active molecule through the dermal layers of skin.

5. The method of claim 1, wherein the moisturizing agents to optimize transdermal absorption comprises agents to improve skin hydration and facilitate transdermal absorption.

6. A method for transdermal delivery of molecules that inhibit, stop, block, or modulate glutaminolysis, glutamine, and other glutaminolysis catalyzing enzymes across multiple metabolic pathways in human beings and animals, the method comprising the step of:
administering said molecules via a transdermal delivery system, wherein the transdermal delivery system is a spray or a mist designed for application to the skin, the spray or a mist comprising a liquid formulation of the spray or mist for delivery of an active molecule, wherein the liquid formulation of the spray or mist comprises specific inhibitors, solvents, surfactants, and stabilizers, and wherein the active molecule is a molecule that inhibits, stops, blocks, or modulates glutaminolysis, glutamine, and other glutaminolysis catalyzing enzymes across multiple metabolic pathways in human beings and animals.

7. The method of claim 6, wherein the active molecule comprises specific inhibitors of glutaminolysis, glutamine, and other glutaminolysis catalyzing enzymes including 6-Diazo-5-oxo-L-norleucine referred to as DON.

8. The method of claim 6, wherein the solvents are selected from a group comprising water, alcohols, and propylene glycol, and wherein alcohols include isopropyl alcohol.

9. The method of claim 6, wherein the surfactants include sodium lauryl sulfate.

10. The method of claim 6, wherein the stabilizers include hydroxypropyl methylcellulose.

11. A method for transdermal delivery of molecules that inhibit, stop, block, or modulate glutaminolysis, glutamine, and other glutaminolysis catalyzing enzymes across multiple metabolic pathways in human beings and animals, the method comprising the step of:
the transdermal delivery system is a transdermal patch system designed for application to the skin for the delivery of an active molecule, wherein the transdermal patch system comprises a backing layer, an adhesive layer, and a reservoir containing the active molecule, and wherein the active molecule is a molecule that inhibits, stops, blocks, or modulates glutaminolysis, glutamine, and other glutaminolysis catalyzing enzymes across multiple metabolic pathways in human beings and animals.

12. The method of claim 11, wherein the active molecule comprises specific inhibitors of glutaminolysis, glutamine, and other glutaminolysis catalyzing enzymes including 6-Diazo-5-oxo-L-norleucine referred to as DON.

13. The method of claim 11, wherein the transdermal patch system adheres to the skin and releases the molecules continuously or intermittently over a specified period.

14. The method of claim 11, wherein the transdermal patch system for optimal adherence and controlled drug release includes materials selected from a group comprising polymeric films, hydrogels, and microneedle arrays, wherein the polymeric films include polyethylene, and wherein the hydrogels include polyacrylic acid-based hydrogels.

15. The method of claim 11, wherein the transdermal patch system further comprises a rate-controlling membrane to control the release kinetics of the inhibitors from the patch.

16. A method for transdermal delivery of molecules that inhibit, stop, block, or modulate glutaminolysis, glutamine, and other glutaminolysis catalyzing enzymes across multiple metabolic pathways in human beings and animals, the method comprising the step of:
the transdermal delivery system is a plug or patch designed for application to the skin for delivery of an active molecule and is implantable in the skin, wherein the plug or patch comprises biocompatible materials and release active molecules from reservoirs comprising an active molecule, wherein the plug or patch is implanted subcutaneously or subdermally, and wherein the active molecule is a molecule that inhibits, stops, blocks, or modulates glutaminolysis, glutamine, and other glutaminolysis catalyzing enzymes across multiple metabolic pathways in human beings and animals.

17. The method of claim 16, wherein the active molecule comprises specific inhibitors of glutaminolysis, glutamine, and other glutaminolysis catalyzing enzymes including 6-Diazo-5-oxo-L-norleucine referred to as DON.

18. The method of claim 16, wherein the biocompatible materials are selected from a group comprising biodegradable polymers including polylactic acid, and hydrogels including alginate.

19. The method of claim 16, wherein the release reservoir provides controlled release of the active molecule over time.

20. The method of claim 16, wherein the administering said molecules via a transdermal delivery system involves implantation procedures involving a minimally invasive technique, including hypodermic needle insertion or surgical implantation.

21. A method for transdermal delivery of molecules that inhibit, stop, block, or modulate glutaminolysis, glutamine, and other glutaminolysis catalyzing enzymes across multiple metabolic pathways in human beings and animals, the method comprising the steps of:
the transdermal delivery system is a transdermal injection or subdermal injection designed for application to the skin for delivery of an active molecule, wherein transdermal injection or subdermal injection comprises a delivery device, and wherein the active molecule is a molecule that inhibits, stops, blocks, or modulates glutaminolysis, glutamine, and other glutaminolysis catalyzing enzymes across multiple metabolic pathways in human beings and animals.

22. The method of claim 21, wherein the active molecule comprises specific inhibitors of glutaminolysis, glutamine, and other glutaminolysis catalyzing enzymes including 6-Diazo-5-oxo-L-norleucine referred to as DON.

23. The method of claim 21, wherein the delivery device is selected from a group of specialized devices comprising microneedles, cannulas, and biodegradable depots for precise and controlled delivery into the subcutaneous tissue.

24. The method of claim 21, wherein the delivery device allows direct administration into the subcutaneous tissue of the skin for sustained release.
